(19) United States
(12) Patent Application Publication
(10) Pub. No.: US 2026/0053359 A1
(43) Pub. Date: Feb. 26, 2026

(54) DEVICE AND METHOD OF MEASURING PROPERTIES OF TARGET

(71) Applicant: ICARE FINLAND OY, Vantaa (FI)

(72) Inventor: Jussi Pukki, Helsinki (FI)

(73) Assignee: ICARE FINLAND OY, Vantaa (FI)

(21) Appl. No.: 19/103,285

(22) PCT Filed: Sep. 14, 2023
(86) PCT No.: PCT/FI2023/050522

§ 371 Date: Feb. 12, 2025

(30) Foreign Application Priority Data
Oct. 5, 2022 (FI) 20225896

(51) Int. Cl. A61B 3/16

(52) U.S. Cl. CPC A61B 3/16

(57) ABSTRACT

A device for measuring properties of a target includes a housing; a probe including an elongated body having a first end and a second end, the first end residing inside the housing and the second end protruding out from the housing during a measurement, the second end including a probe head, the probe configured to impact the target with the probe head; a sensor system, including one or more distance sensors located on the housing to measure one or more distances between the one or more distance sensors and one or more locations on the target a probe attachment means to retain the probe within the housing; and a probe release means to release the probe towards the target if each of the one or more distances between the one or more distance sensors and one or more locations on the target is within a predefined range from a predefined value.

### TECHNICAL FIELD

The present disclosure relates to a device of measuring properties of a target. The present disclosure also relates to a method of measuring properties of a target.

### BACKGROUND

Over the past few millennia, measurement devices have gained popularity in various disciplines such as medicine, engineering, and the like. Particularly, in the medicine of eyes, the target is an eye or a part of an eye of a measurement subject, and a measurement device is employed to measure a property of the target, such as intraocular pressure, dimensions of the eyeball such as eyeball length, thickness of lens, thickness of cornea, tactile sensitivity and so forth, by medical professionals such as medical doctors, optometrists, ophthalmologists, and the like, in order to measure parameters and/or to diagnose ailments associated with the said targets.

Conventionally, such measurements have been performed with measurement instruments, that require their probe to be pressed against the target for a substantially long time with a substantially high force, that necessitates use of local anaesthesia to avoid causing pain to the measurement subject. Recently, rebound measuring devices have been available to measure several properties with a much smaller force and shorter contact time, thus making use of local anaesthesia unnecessary.

As an example of rebound measuring devices, a rebound tonometer may be employed to measure a fluid pressure inside an eye, i.e. an intraocular pressure, by interacting with cornea of the eye to an indentation.

As another example, a rebound esthesiometer may be used to measure touch sensitivity of a target such as an eye, i.e. a minimum impact that can be felt by the measurement subject.

As another example, a rebound dimensions measuring instrument may be used to measure dimensions based on e.g. a propagation time of an acoustic wave, of various parts of a target such as an eye, while a head of a rebound probe is in contact with the target.

Generally, when using such rebound device, the rebound device is arranged in front of the target at a particular position, including a particular distance from the target. It is challenging to put the device in said position and distance.

There exist very specific requirements of alignment of a rebound device with respect to a target, in order to measure the parameters associated with the target. In some existing cases, a rebound device is positioned with mechanical means to a distance of a forehead. Due to anatomy variation, it might be arranged too far or too close from the target. In such cases, the parameters are inaccurately measured or are not measured at all. When the rebound device is arranged too close to the target, part(s) of said device could unintentionally harm the subject while measuring the target parameters.

Some of existing rebound measuring devices comprise rests for contacting the surroundings of a target in order to facilitate arranging of the rebound measuring device correctly in front of the target. The rests define a distance and inclination between the rebound measuring device and the target. However, due to anatomy variation the device might be arranged too far or too close from the target or aimed to a wrong position on the target. This is because said rests are mechanically fixed on the rebound tonometer, and are arranged manually with respect to the target. Some of existing rebound measuring devices must be arranged with respect to the target manually, without even such rests.

The existing rebound measuring devices require the subject to be fully attentive or alert during the measurement, otherwise the measurement accuracy and efficiency would be affected. Therefore, in light of the foregoing discussion, there exists a need to overcome the aforementioned drawbacks associated with existing means for measuring properties of the target.

### SUMMARY

The present disclosure seeks to provide a device of measuring properties of a target. The present disclosure also seeks to provide a method of measuring properties of a target. An aim of the present disclosure is to provide a solution that overcomes at least partially the problems encountered in prior art.

In one aspect, an embodiment of the present disclosure provides a device of measuring properties of a target, the device comprising:
a housing,
a probe comprising an elongated body having a first end and a second end, the first end residing inside the housing and the second end protruding out from the housing during a measurement, the second end comprising a probe head, the probe operable to impact the target with the probe head,
a sensor system comprising one or more distance sensors located on the housing, the one or more distance sensors being operable to measure one or more distances (L) between the one or more distance sensors and one or more locations on the target, the one or more distances being parallel to a direction of the elongated body axis,
a probe attachment means operable to retain the probe within the housing; and
a probe release means operable to release the probe towards the target if each of the one or more distances between the one or more distance sensors and one or more locations on the target is within a predefined range (+/−R) from a predefined value (V), characterized in that, the one or more distance sensors comprise:
   a first distance sensor operable to measure a first distance (L1) between the first distance sensor and a first location on the target, and
   a second distance sensor operable to measure a second distance (L2) between the second distance sensor and a second location on the target,
wherein the probe release means is operable to release the probe towards the target if the first distance (L1) is within a predefined range (+/−R) from a first predefined value (V1) and the second distance (L2) is within a predefined range from a second predefined value (V2).

In another aspect, an embodiment of the present disclosure provides a method of measuring properties of a target, the method comprising:
measuring one or more distances (L) between one or more distance sensors and one or more locations on the target, and
releasing towards the target a probe if each of said one or more distances (L) between one or more distance sensors and one or more locations on the target is within a predefined range (+/−R) from a predefined value (V),
wherein the probe comprises an elongated body having a first end and a second end, the first end residing inside the housing and the second end protruding out from the housing during a measurement, the second end comprising a probe head, and
wherein the one or more distances (L) is being parallel to a direction of the elongated body axis,
characterized in that, measuring one or more distances (L) comprise:
   measuring a first distance (L1) between a first distance sensor and a first location on the target,
   measuring a second distance (L2) between a second distance sensor and a second location on the target,
   releasing the probe towards the target if the first distance (L1) is within a predefined range (+/−R) from a first predefined value (V1), and the second distance (L2) is within a predefined range (+/−R) from a second predefined value (V2).

Embodiments of the present disclosure substantially eliminate or at least partially address the aforementioned problems in the prior art, and enable an improved, accurate, reliable and efficient device of measuring properties of a target. Beneficially, the device enables an automatic release of the rebound probe, to obtain measurement of the properties of a target, when distance between thereof and the target is known to be acceptable, and if three distance sensors are used, also aiming and inclination of the device are known to be acceptable. Moreover, the device eliminates a need of frequent (or constant) human intervention.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a device of measuring properties of a target, in accordance with an embodiment of the present disclosure;

FIG. 2 is a front view of a device of measuring properties of a target, in accordance with an embodiment of the present disclosure;

FIG. 3 is an exploded view of a device of measuring properties of a target, in accordance with an embodiment of the present disclosure;

FIG. 4A and FIG. 4B are a perspective view of a device of measuring properties of a target, in accordance with an embodiment of the present disclosure; and

FIG. 5 is a flowchart depicting steps of a method of measuring properties of a target, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides a device of measuring properties of a target, the device comprising:
a housing,
a probe comprising an elongated body having a first end and a second end, the first end residing inside the housing and the second end protruding out from the housing during a measurement, the second end comprising a probe head, the probe operable to impact the target with the probe head,
a sensor system comprising one or more distance sensors located on the housing, the one or more distance sensors being operable to measure one or more distances (L) between the one or more distance sensors and one or more locations on the target, the one or more distances being parallel to a direction of the elongated body axis,
a probe attachment means operable to retain the probe within the housing; and
a probe release means operable to release the probe towards the target if each of the one or more distances between the one or more distance sensors and one or more locations on the target is within a predefined range (+/−R) from a predefined value (V); characterized in that, the one or more distance sensors comprise:
   a first distance sensor operable to measure a first distance (L1) between the first distance sensor and a first location on the target, and
   a second distance sensor operable to measure a second distance (L2) between the second distance sensor and a second location on the target,
wherein the probe release means is operable to release the probe towards the target if the first distance (L1) is within a predefined range (+/−R) from a first predefined value (V1) and the second distance (L2) is within a predefined range from a second predefined value (V2).

In another aspect, an embodiment of the present disclosure provides a method of measuring properties of a target, the method comprising:
measuring one or more distances (L) between one or more distance sensors and one or more locations on the target, and
releasing towards the target a probe if each of said one or more distances (L) between one or more distance sensors and one or more locations on the target is within a predefined range (+/−R) from a predefined value (V),
wherein the probe comprises an elongated body having a first end and a second end, the first end residing inside the housing and the second end protruding out from the housing during a measurement, the second end comprising a probe head, and
wherein the one or more distances (L) is being parallel to a direction of the elongated body axis,
characterized in that, measuring one or more distances (L) comprise:
   measuring a first distance (L1) between a first distance sensor and a first location on the target,
   measuring a second distance (L2) between a second distance sensor and a second location on the target,
   releasing the probe towards the target if the first distance (L1) is within a predefined range (+/−R) from a first predefined value (V1), and the second distance (L2) is within a predefined range (+/−R) from a second predefined value (V2).

The present disclosure provides the aforementioned device and the aforementioned method that is robust, fast, effective, reliable and user friendly. Beneficially, the device employs the sensor system that enables an automatic probe release, to automatically or manually start measurement of the properties of the target, when position thereof in respect of the target is acceptable. Advantageously, the device does not require the subject to be fully attentive or alert during the measurement, thus not affecting the measurement accuracy of the device. Furthermore, the aforementioned device significantly speeds up the measurement of the target and provides a more reliable estimation thereof.

Pursuant to the embodiments of the present disclosure, the term “target” as used herein refers to an element of a body, such as a human body, an animal body or an industrial product. When referred as human or animal body, the element being such as a body part whose property is to be measured.

Optionally, the target is an eye. Dimensions of several parts of the eye may be measured, such as of an eyeball, cornea, iris, pupil, aqueous humor, lens, vitreous humor, retina, and optic nerve. The threshold of the corneal sensitivity of the eye may be measured (esthesiometry). Intra ocular pressure (IOP) may be measured (tonometry).

The term “intra ocular pressure” (IOP) as used herein refers to a fluid pressure of the eye. Pressure is a measure of force per unit area, the IOP is a measurement that involves the magnitude of the force exerted by the aqueous humor on an internal surface area of the eyeball.

Notably, the measurement of the intraocular pressure is of vital importance to maintain overall eye health and function. The measurement of the intraocular pressure enables a diagnosis and treatment of ocular hypertension before the development of eye-related conditions such as conjunctivitis, corneal infections, glaucoma, dry-eye, and so forth.

The term “corneal sensitivity” as used herein refers to a smallest impact force that a subject can notice, when repeatedly bombarded with a rebound probe using a set of impact forces.

The device comprises a housing. The term “housing” as used herein refers to a protective layer that is configured to surround various components of the device completely or at least partly. In other words, the housing is adapted to accommodate the components of the device. It will be appreciated that the components of the device could be arranged (namely, held or attached) in the housing via chemical means, mechanical means, magnetic means, and the like. In an implementation, the components of the device could be manufactured individually, and then assembled in the housing. In another implementation, the components of the device could be manufactured as an integral part of the housing.

Moreover, the device comprises a probe. The term “probe” as used herein refers to a tool employed for determining properties of the target. In this regard, the probe has an elongated body. Furthermore, the elongated body has a first end, that is inside the housing of the device while measurement is done. Additionally, the elongated body has a second end, that protrudes outside the housing of the device when in use. The term “probe head” as used herein refers to a thicker part of the probe, at the second end of the probe. This may be an active probe head that contains sensor electronics or a passive probe head. Beneficially, the probe head increases surface area of the contact between target and probe, thereby reducing an impact pressure of the probe on the ocular surface. Optionally, the probe is designed in a manner to have smaller contact and higher impact with the ocular surface of the eye.

Optionally, the probe is a disposable probe in order to prevent contamination of the target. Optionally, the probe head is made from a bio-compatible material in order to prevent contamination of the target.

The term “sensor system” as used herein refers to an arrangement that comprises sensors, which are used to detect, track, or measure a distance between various objects. In this regard, the sensor system comprises one or more distance sensors that are located on the housing. It will be appreciated that the one or more distance sensors be operable to measure a distance between the one or more distance sensors and one or more locations on the target. Advantageously, the sensor system ensures that, when in operation, the distance is parallel to a direction of the elongated body axis, thus enabling an accurate measurement of the properties of the target.

Optionally, the sensor system further comprises one or more sensors located in the probe head, wherein the probe is an active probe, the one or more sensors being operable to measure one or more properties of the target. In another embodiment, wherein the probe is a passive probe, the one or more sensors are located in the housing, the one or more sensors being operable to measure one or more properties of the target. The properties may comprise at least one of selected from intra ocular pressure, dimensions of one or more parts of an eyeball, tactile sensitivity. When the correct position of the device and the probe head regarding the target is detected, the probe is released towards the target to measure the properties of the target. This enables easy and more accurate measurements. For example, if the target is an eye, the one or more sensors are operable to measure intra ocular pressure, dimensions of one or more parts of an eyeball and tactile sensitivity.

Optionally, the one or more distance sensor comprises at least one optical sensor that is configured to measure a deflection of an electromagnetic beam, such as visible or invisible light, reflected from the surface of the target. Herein, a light beam sensor refers to an electronic device that measures the distance of an object by emitting a light beam, and converting the deflection of the reflected light beam into an electrical signal.

Optionally, the one or more distance sensor comprises at least one optical sensor that is configured to measure a time of flight of an electromagnetic pulse reflected from the surface of the target. Herein, a time-of-flight sensor refers to an electronic device, that measures the distance of an object by emitting pulse, and converting the time between transmit pulse and reflected pulse into an electrical signal.

Optionally, the one or more distance sensors comprises at least one of selected from ultrasonic sensor, electromagnetic sensor. Herein, an ultrasonic sensor refers to an electronic device that measures the distance of an object by emitting ultrasonic sound waves, and converts the reflected sound into an electrical signal. Typically, an ultrasonic sensor comprises piezoelectric crystal(s), coil(s) and membrane(s) or MEMS(s), to emit a sound, and receive the sound returning from a target. In an implementation, the sensor measures the time taken between the emission and the reception of the sound in order to calculate the distance between the sensor and the target.

In this regard, an ultrasonic sensor or an electromagnetic sensor may be employed in the housing of the device to obtain distance (L), as well as in the probe head to obtain the target properties.

Optionally, the probe head is configured to perform parameter measurement coincident with the contact between the probe head and the target, i.e. at a fixed delay from the probe launch, as the distance and the flight time of the probe over the said distance is controlled, and thus known.

Furthermore, the housing comprises a probe attachment means operable to retain the probe within the housing. The term “probe attachment means” as used herein refers to a magnetic, mechanical or other type of means, that is used to hold the probe in a certain position within the housing before releasing of the probe to fly towards a target, and, optionally, capture the probe after rebounding of the probe.

Optionally, the probe attachment means prevents the probe from dropping off of the device. Optionally, the probe attachment means is strategically designed to allow friction free movement of the probe along a longitudinal axis of the probe when in operation. Optionally, the probe attachment means comprises a mechanical lock, a frictional brake, an induction coil, an electrical conductor (for example, a wire) in shape of a coil, spiral, helix, a permanent magnet, and the like. Optionally, the probe attachment means induces an electric field, a frictional force and/or a magnetic field into the probe for its retention in the housing of the device.

The housing comprises a probe release means. The term “probe release means” as used herein refers to a magnetic, mechanical or other type of means, that is used to release the probe from the housing in a certain position and a certain direction, in a controlled manner. In this regard, the probe release means enables an efficient and accurate movement of the probe, when in use.

The term “predefined value” as used herein refers to a measurable quantity such as a distance, a force, an acceleration, a time etc. that is predetermined in order to achieve accurate results, when in operation. In this regard, the probe release means is operable to release the probe towards the target if each of the one or more distances between the one or more distance sensors and one or more locations on the target is within a predefined range (+/−R) from a predefined value (V). This means, that each of one or more distances must be in the same range from a predefined value (V) to release the probe. Otherwise, if distances are not in the same range, the angle for releasing the probe is incorrect. The range R is predetermined by assessment of distance measuring inaccuracy of the embodiment.

It will be appreciated that the said arrangement may also protect the target from damage.

Optionally, the probe release means may be air pressure, spring, electromagnetic induction, piezoelectric force, and the like.

Herein, “air pressure” refers to a force per unit area. If gas is pumped in a vessel, the pressure in the vessel increases and stores mechanical energy, that can be then used to release the probe.

The term “spring” as used herein refers to an elastic machine element, that possesses an ability to deflect under an action of a load and returns to an original shape when the load is removed. In other words, the spring is an elastic object that stores mechanical energy.

The term “electromagnetic induction” as used herein refers to producing a mechanical force from an electromagnetic field, or an electromagnetic field from a mechanical force. In an implementation, an induction coil may be arranged around the elongated body of the probe to provide motion to the probe, when in use. Optionally, the induction coil is used for measuring speed and acceleration of the probe, when ejected, touches the target and bounces back.

The term “piezoelectric force” as used herein refers to producing a mechanical force and deformation in a machine element by applying a voltage upon one or more parts made of a piezoelectric material.

Optionally, the elongated body is a pin. Optionally, the elongated body is made of magnetizable material. Optionally the said magnetizable material comprises ferromagnetic metal. Optionally the elongated body has been magnetized before release.

Optionally, the elongated body is a magnetic pin and the probe release means is operable to release the probe towards the target by driving current to the probe release means, and the driving current waveform defines the impact parameters. In an example, the pin may be made of a thin wire of magnetic material. Optionally, the magnetic material in the magnetic pin may be ferromagnetic. In this regard, optionally, the current causes the probe release means to generate an electromagnetic field. Moreover, the generated electromagnetic field generates a controlled force to the probe in order to control the movement thereof for measuring the properties of the target.

The one or more distance sensors comprise:
a first distance sensor operable to measure a first distance (L1) between a first distance sensor and a first location on the target, and
a second distance sensor operable to measure a second distance (L2) between the second distance sensor and a second location on the target, wherein the probe release means are operable to release the probe towards the target if
the first distance (L1) is within a predefined range from a predefined value (V1) and the second distance (L2) is within a predefined range from a predefined value (V2). the first distance (L1) is within a predefined range (+/−R) from a first predefined value (V1) and the second distance (L2) is within a predefined range from a second predefined value (V2).

Such implementation of the first distance sensor and the second distance sensor ensures that at least two parallel distances are measured at at least two locations on target, namely the first location and the second location, and it is more accurate to determine the angle and distance for releasing the probe towards the target in desired position. Such an implementation enables to avoid an inclination error in a horizontal axis. This ensures, that measurements of the properties of the target are carried out more effectively.

Throughout the present disclosure, the term “first distance sensor”, “second distance sensor” and “third distance sensor” as used herein refers to distance or displacement sensors that are used for determining the distance of an object from another object without a physical contact.

Optionally, the first distance sensor, the second distance sensor and the third distance sensor are implemented as an ultrasonic sensor that measures the distance between the target and the device through a value of output voltage, output current or output time between transitions thereof. Optionally, the output value is generated as a result of ultrasonic sound waves. In an embodiment, the first distance sensor and the second distance sensor are employed on the housing of the device in such a way that the first distance sensor and the second distance sensor is arranged in line with the probe head. In another embodiment the first distance sensor, the second distance sensor and the third distance sensor are employed on the housing of the device in such a way that all three are located at equal distances of each other. It will be appreciated that the said arrangement enables the device to be positioned at an optimal distance from the target such as the eye.

In an implementation, the probe release means are operable to release the probe towards the target when the distance measured between the first distance sensor and the first location on the target is within a predefined range (+/−R) from a predefined value (V) in order to enable the automatic and smooth ejection of the probe towards the target. Optionally, the probe release means operable to release the probe is selected from at least one of from automatic or manual. Subsequently, the probe release means is operable to release the probe towards the target when the distance measured between each distance sensor and each corresponding location on the target is within a predefined range (+/−R) from a predefined value (V) in order to position the device at the distance and inclination favorable to taking measure, and by the device to wait until it is at a favorable position and then launch the probe automatically or manually towards the target. The favorable position is defined by desired distance (L) and inclination in 3-axis. Optionally, advantageously, the measured distance values are used by an operator to position the device at a position favorable to taking measure, and by the device to wait until it is at a favorable position and then eject the probe automatically.

To release the probe manually towards the target, the device may comprise a button or other similar activation device to manually start the release of the probe. For manual release, the probe release means is operable to block the manually releasing when one or more distance is not in the predefined range from a predefined value. This means, that for example, when pushing the button when the predefined range of one or more distances is not achieved, the probe will not be released. However, once the predefined range of each distance is achieved, the probe can be released towards the target by pushing the button or any other similar manual activation mechanism.

Optionally, the one or more distance sensors comprises
a first distance sensor operable to measure a first distance (L1) between a first distance sensor and a first location on the target, and
a second distance sensor operable to measure a second distance (L2) between the second distance sensor and a second location on the target,
a third distance sensor operable to measure a third distance (L3) between the third distance sensor and a third location on the target,
wherein the probe release means are operable to release the probe towards the target if
the first distance (L1) is within a predefined range (+/−R) from a first predefined value (V1), the second distance (L2) is within a predefined range from a second predefined value (V2) and the third distance (L3) is within a predefined range from a third predefined value (V3) and
wherein the first distance sensor, the second distance sensor and the third distance sensor are arranged at corners of a triangular shape.

Such implementation of measuring at least three parallel distances between at least three locations on target, namely the first location, the second location and the third location, and the first distance sensor, the second distance sensor, the third distance sensor ensures more accurate correct angle and distance for releasing the probe towards the target in desired position.

In this regard, the sensor system comprises the first distance sensor, the second distance sensor and the third distance sensor to measure the distance between thereof and the corresponding first location, the second location and the third location, respectively on the surface of the eye. It will be appreciated that the first distance sensor, the second distance sensor and the third distance sensor are arranged at corners of a triangular shape. Moreover, when measuring the properties of the target, the sensor system reduces variability of the probe ejection time and the probe impact energy.

Optionally, the probe sensor system further comprises a timer operable to be set based on the measured distance (L) and a known or measured speed of the probe to start measurement of the properties of the target. The term “timer” as used herein refers to an instrument that measures time, especially one that is part of a machine and causes start or stop of working thereof at predetermined intervals of time. In this regard, the time interval is set based on the measured distance (L) and the known flight time of the probe to start measurement of the properties of the target. Optionally, the timer may be associated with the active probe head.

Optionally the active probe head or the sensor system comprises a timer set to a predetermined value based on the known flight time of the probe, and the sensor system is communicatively coupled with the active probe head, to enable the active probe head to time parameter measurement taking correctly, to coincide with the time of physical contact between the probe head and the target. Optionally, mechanical waves, e.g. ultrasound, are sent to the target based on the ejection time of the probe and the known probe flight time, thus eliminating the need of other means to trigger the measurement. Optionally, electromagnetic pulses of visible wavelength are sent to the target based on the ejection time of the probe and the known probe flight time, thus eliminating the need of other means to trigger the measurement. Optionally, electromagnetic pulses of non-visible wavelength are sent to the target based on the ejection time of the probe and the known probe flight time, thus eliminating the need of other means to trigger the measurement.

Optionally, the first distance sensor, the second distance sensor and the third distance sensor are arranged at equal distances from each other. In this regard, the said arrangement enables the first distance sensor, the second distance sensor and the third distance sensor to obtain a triangular measurement of the properties of the target. It will be appreciated that the said arrangement of the first distance sensor, the second distance sensor and the third distance sensor in the triangular shape provides a full coverage to the target distance, inclination and centering, when measuring the properties thereof. Beneficially, the operator of the device may measure the properties of the target precisely from various angles. Optionally, the first distance sensor, the second distance sensor and the third distance sensor allow the device to be kept upright or at other predefined inclination, and within the appropriate distance from the corneal center of the eye.

The present disclosure also relates to the method as described above. Various embodiments and variants disclosed above apply mutatis mutandis to the method.

The present disclosure relates to a method of measuring properties of a target, the method comprising:
measuring one or more distances (L) between one or more distance sensors and one or more locations on the target, and
releasing towards the target a probe if each of said one or more distances (L) between one or more distance sensors and one or more locations on the target is within a predefined range (+/−R) from a predefined value (V),
wherein the probe comprises an elongated body having a first end and a second end, the first end residing inside the housing and the second end protruding out from the housing during a measurement, the second end comprising a probe head, and
wherein the one or more distances (L) is being parallel to a direction of the elongated body axis,
characterized in that, measuring one or more distances comprise:
   measuring a first distance (L1) between a first distance sensor and a first location on the target,
   measuring a second distance (L2) between a second distance sensor and a second location on the target,
   releasing the probe towards the target if the first distance (L1) is within a predefined range (+/−R) from a first predefined value (V1), and the second distance (L2) is within a predefined range (+/−R) from a second predefined value (V2).

Measuring one or more distances comprise
measuring a first distance (L1) between a first distance sensor and a first location on the target,
measuring a second distance (L2) between a second distance sensor and a second location on the target,
releasing the probe towards the target if the first distance (L1) is within a predefined range (+/−R) from a first predefined value (V1), and the second distance (L2) is within a predefined range (+/−R) from a second predefined value (V2).

In another embodiment measuring one or more distances comprise
measuring a first distance (L1) between a first distance sensor and a first location on the target,
measuring a second distance (L2) between a second distance sensor and a second location on the target,
measuring a third distance (L3) between a third distance sensor and a third location on the target,
releasing the probe towards the target if the first distance (L1) is within a predefined range (+/−R) from a first predefined value (V1), the second distance (L2) is within a predefined range (+/−R) from a second predefined value (V2) and the third distance (L3) is within a predefined range (+/−R) from a third predefined value (V3), and
wherein the first distance sensor, the second distance sensor and the third distance sensor are arranged at corners of a triangle.

Optionally, the method may further comprise starting measuring properties of the target based on the measured one or more distances and a known flight time of the probe.

Optionally, the method may further comprise measuring one or more properties of the target using sensors when the probe head is in mechanical contact with target, the sensors being located in at least one location selected from the probe head and the housing, wherein the properties of the target comprise at least one of selected from intra ocular pressure, one or more dimensions of an eyeball, tactile sensitivity.

### DETAILED DESCRIPTION OF THE DRAWINGS

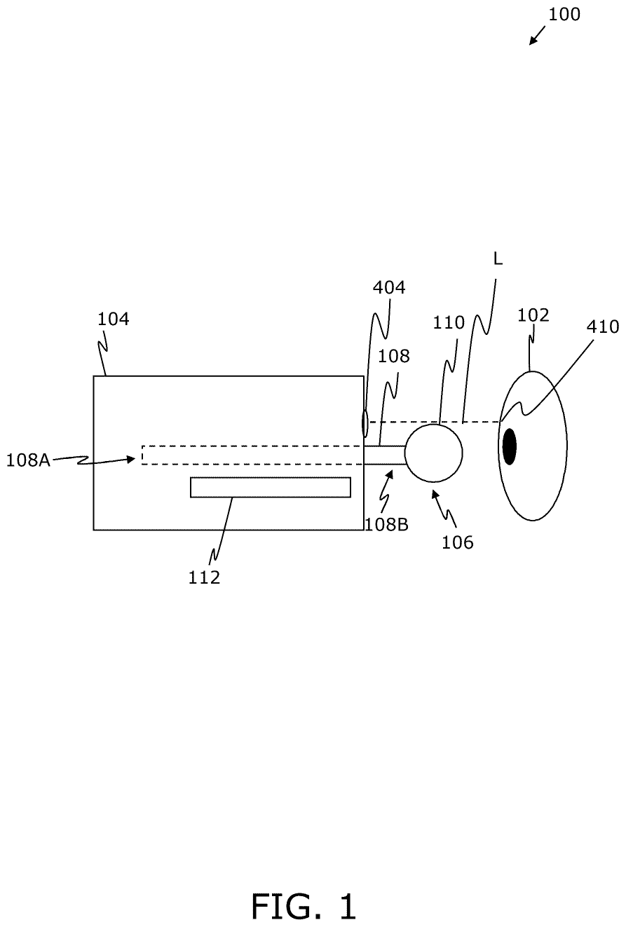

Referring to FIG. 1, shown is a schematic illustration of device 100 of measuring properties of a target 102, in accordance with an embodiment of the present disclosure. Herein, the device 100 comprises a housing 104 and a probe 106. Moreover, the probe 106 comprises an elongated body 108 having a first end 108A and a second end 108B, the first end 108A resides inside the housing 104 and the second end 108B protrudes out from the housing 104 during measurement, the second end 108B comprising a probe head 110, the probe 106 operable to impact the target 102 with the probe head 110. Furthermore, the housing 104 comprises a probe base means 112 operable to retain the probe within the housing 104 and to release the probe 106 in a controlled manner towards the target 102. One or more distance sensors 404 are located on the housing to measure one or more distances (L) between the one or more distance sensors 404 and one or more locations 410 on the target 102.

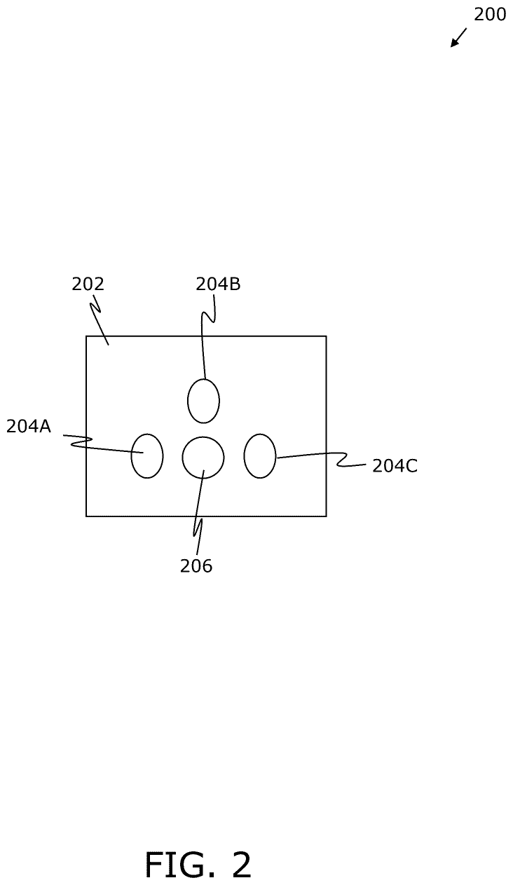

Referring to FIG. 2, shown is a front view of a device 200 of measuring properties of a target (not shown), in accordance with an embodiment of the present disclosure. As shown, the device 200 comprises a housing 202 comprising a probe head 206, a sensor system 204 comprising one or more distance sensors located on the housing 202, the one or more distance sensors being operable to measure a distance (not shown) between the one or more distance sensors and one or more locations on the target, the distance being parallel to a direction of the elongated body axis. As shown, the sensor system 204 comprises a first distance sensor 204A, a second distance sensor 204B, and a third distance sensor 204C. Moreover, the first distance sensor 204A, the second distance sensor 204B and the third distance sensor 204C are arranged at corners of a triangular shape.

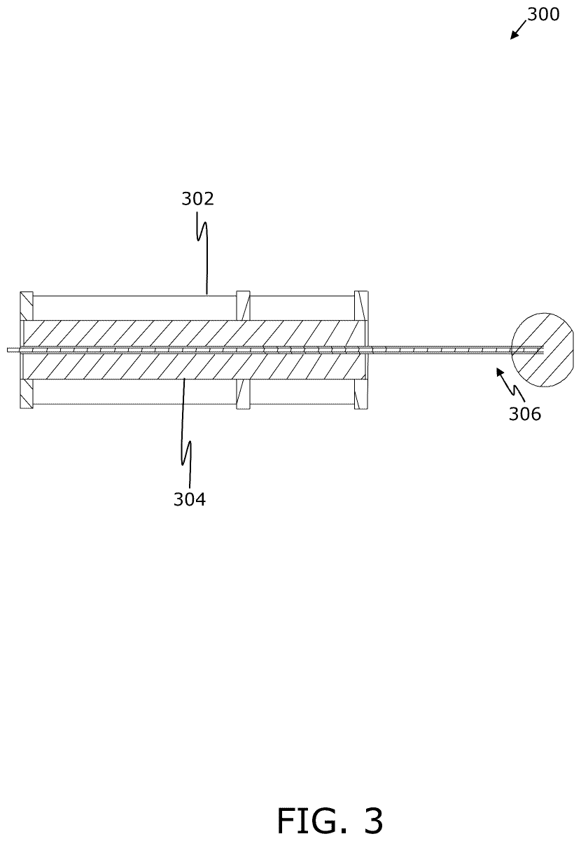

Referring to FIG. 3, shown is an exploded view of a device 300 of measuring properties of a target (not shown), in accordance with an embodiment of the present disclosure. As shown, the device 300 comprises a probe base 302 inside a housing (not shown). Moreover, the probe base 302 comprises a probe release means 304 operable to release a probe 306 towards the target in a controlled manner if the distance between each of the one or more distance sensors (not shown) and one or more locations on the target is within a predefined range from a predefined value V.

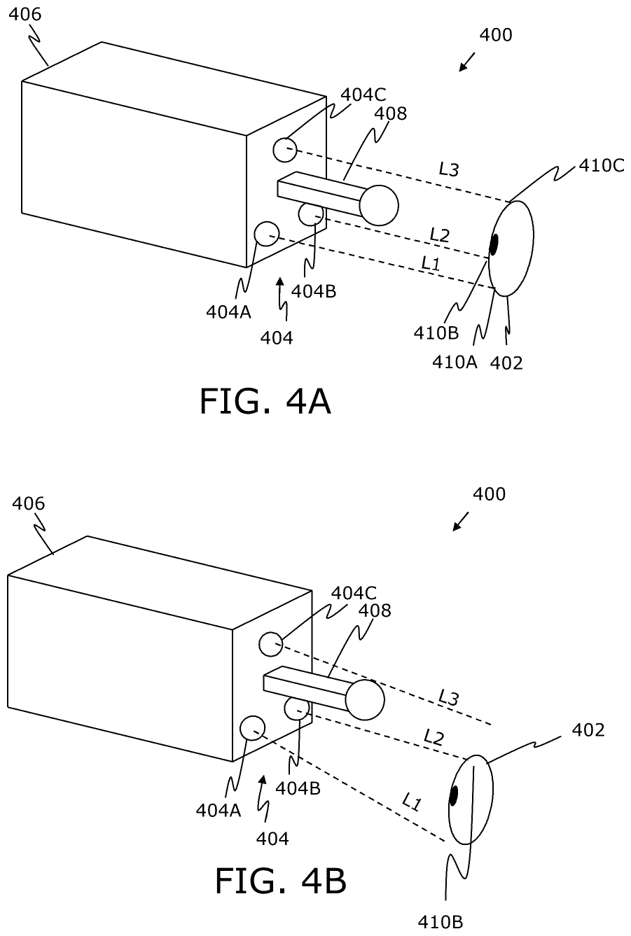

Referring to FIG. 4A and FIG. 4B, shown are a perspective view of a device 400 of measuring properties of a target 402, in accordance with an embodiment of the present disclosure. As shown in FIG. 4A, the device 400 is arranged at an optimum angle with respect to the target 402. Herein, the device 400 comprises a sensor system 404 located on a housing 406. Moreover, the sensor system 404 comprises a first distance sensor 404A operable to measure the distance between the first distance sensor 404A and a first location 410A on the target 402, a second distance sensor 404B operable to measure the distance between the second distance sensor 404B and a second location 410B on the target 402, a third distance sensor 404C operable to measure the distance between the third distance sensor 404C and a third location 410C on the target 402, wherein a probe release means (not shown) are operable to release a probe 408 towards the target 402 if the distance L1 between the first distance sensor 404A and a first location on the target 402 is within a predefined range from a predefined value V1, the distance L2 between the second distance sensor 404B and a second location on the target 402 is within a predefined range from a predefined value V2 and the distance L3 between the third distance sensor 404C and a third location on the target 402 is within a predefined range from a predefined value V3. It will be appreciated that the first distance sensor 404A, the second distance sensor 404B and the third distance sensor 404C are arranged at corners of a triangle.

As shown in FIG. 4B, the device 400 is arranged at an inaccurate angle with respect to the target 402, thereby leading to an inaccurate measuring of the properties of the target 402. Herein, at least one of the distance L1 between the first distance sensor 404A and the first location on the target 402, the distance L2 between the second distance sensor 404B and the second location on the target 402 and the distance L3 between the third distance sensor and the third location on the target 402 is not within predefined range of the corresponding predefined value, causing the probe 408 to not hit the intended spot on the surface of the target, or the flight time to be too long or too short.

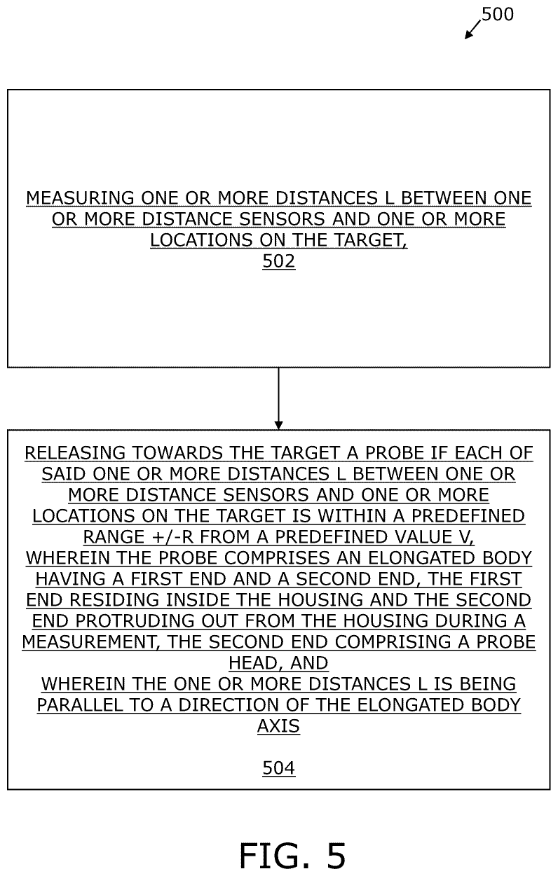

Referring to FIG. 5, shown is a flowchart depicting steps of a method 500 of measuring properties of a target, in accordance with an embodiment of the present disclosure. At step 502, one or more distances L is measured between one or more distance sensors and one or more locations on the target.

At step 504, a probe is released towards the target if each of said one or more distances L between one or more distance sensors and one or more locations on the target is within a predefined range +/−R from a predefined value V,
wherein the probe comprises an elongated body having a first end and a second end, the first end residing inside the housing and the second end protruding out from the housing during a measurement, the second end comprising a probe head, and
wherein the one or more distances L is being parallel to a direction of the elongated body axis.

The steps 502, and 504 are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A device for measuring properties of a target the device comprising:
a housing,
a probe comprising an elongated body having a first end and a second end, the first end residing inside the housing and the second end protruding out from the housing during a measurement, the second end comprising a probe head, the probe configured to impact the target with the probe head,
a sensor system comprising one or more distance sensors located on the housing, the one or more distance sensors being operable to measure one or more distances between the one or more distance sensors and one or more locations on the target, the one or more distances being parallel to a direction of the elongated body axis,
a probe attachment means configured to retain the probe within the housing; and
a probe release means configured to release the probe towards the target if each of the one or more distances between the one or more distance sensors and one or more locations on the target is within a predefined range from a predefined value;
wherein that, the one or more distance sensors comprise:
a first distance sensor configured to measure a first distance between the first distance sensor and a first location on the target, and
a second distance sensor configured to measure a second distance between the second distance sensor and a second location on the target,
wherein the probe release means is configured to release the probe towards the target if the first distance is within a predefined range from a first predefined value and the second distance is within a predefined range from a second predefined value.

2. The device according to claim 1, wherein the one or more distance sensors further comprise:
a third distance sensor configured to measure a third distance between the third distance sensor and a third location on the target,
wherein the probe release means is configured to release the probe towards the target if
the first distance is within a predefined range from a first predefined value the second distance is within a predefined range from a second predefined value and the third distance is within a predefined range from a third predefined value and
wherein the first distance sensor, the second distance sensor and the third distance sensor are arranged at corners of a triangular shape.

3. The device according to claim 2, wherein the first distance sensor, the second distance sensor and the third distance sensor are arranged at equal distances from each other.

4. The device according to claim 1, wherein the sensor system further comprises a timer configured to be set based on a measured distance and a known travel time of the probe to start measurement of the properties of the target.

5. The device according to claim 1, wherein the target is an eye.

6. The device according to claim 1, wherein the properties of the target comprise at least one of selected from intra ocular pressure, one or more dimensions of at least a part of an eyeball, tactile sensitivity.

7. The device according to claim 1, wherein the sensor system comprises at least one of selected from ultrasonic sensor, electromagnetic sensor.

8. The device according to claim 1, wherein the sensor system further comprises:
one or more sensors located in the probe head, wherein the probe is an active probe, the one or more sensors being configured to measure one or more properties of the target.

9. The device according to claim 1, wherein the sensor system further comprises:
one or more sensors located in the housing, wherein the probe is a passive probe, the one or more sensors being configured to measure one or more properties of the target.

10. The device according to claim 1, wherein the probe release means configured to release the probe is selected from at least one of from automatically or manually.

11. The device according to claim 10, wherein the probe release means is configured to block the manually releasing when one or more distance is not within a predefined range from the predefined value.

12. A method of measuring properties of a target, the method comprising:
measuring one or more distances between one or more distance sensors and one or more locations on the target,
and
releasing towards the target a probe if each of said one or more distances between one or more distance sensors and one or more locations on the target is within a predefined range from a predefined value,
wherein the probe comprises an elongated body having a first end and a second end, the first end residing inside the housing and the second end protruding out from the housing during a measurement, the second end comprising a probe head and
wherein the one or more distances is being parallel to a direction of the elongated body axis,
wherein that, measuring one or more distances comprise:
measuring a first distance between a first distance sensor and a first location on the target,
measuring a second distance between a second distance sensor and a second location on the target,
releasing the probe towards the target if the first distance is within a predefined range from a first predefined value and the second distance is within a predefined range from a second predefined value

13. The method according to claim 12, wherein measuring one or more distances comprise:
measuring a third distance between a third distance sensor and a third location on the target,
releasing the probe towards the target if the first distance is within a predefined range from a first predefined value, the second distance is within a predefined range from a second predefined value and the third distance is within a predefined range from a third predefined value, and
wherein the first distance sensor, the second distance sensor and the third distance sensor are arranged at corners of a triangle.

14. The method according to claim 12 further comprising starting measuring properties of the target based on the measured one or more distances and a known flight time of the probe.

15. The method according to claim 12 further comprising:
measuring one or more properties of the target when the probe head is in mechanical contact with the target with sensors located in at least one location selected from the probe head and the housing, wherein the properties of the target comprise at least one of selected from intra ocular pressure, one or more dimensions of an eyeball, tactile sensitivity.
